SEQUENCE LISTING (10 sequences)

SEQ ID NO: 1 - DNA, 12361 bp, Artificial Sequence
  pCAMBIA1303
catggtagatctgactagtttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttcagctgtctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctagccaccaccaccaccaccacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgac

SEQ ID NO: 2 - DNA, 20 bp, Artificial Sequence
  GUS Primer - left
ctgatagcgcgtgacaaaaa

SEQ ID NO: 3 - DNA, 20 bp, Artificial Sequence
  GUS Primer - right
ggcacagcacatcaaagaga

SEQ ID NO: 4 - DNA, 20 bp, Artificial Sequence
  GFP Primer - left
tcaaggaggacggaaacatc

SEQ ID NO: 5 - DNA, 20 bp, Artificial Sequence
  Primer for Genome PCR
aaagggcagattgtgtggac

SEQ ID NO: 6 - DNA, 1812 bp, Artificial Sequence
  GUS gene
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 7 - DNA, 356 bp, Artificial Sequence
  scFv gene (VH)
aggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagaggggcctataaaaggggatatgctatggactactggggtcaaggaacctcagtcaccgtctcc

SEQ ID NO: 8 - DNA, 345 bp, Artificial Sequence
  scFv gene (VL)
tgtgatgtcacagtctccatcctccctggctgtgtcagcaggagagaaggtcactatgagctgcaaatccagtcagagtctgttcaacagtagaacccgaaagaactacttggcttggtaccagcagaaaccagggcagtctcctaaactgctgatctactgggcatccactagggaatctggggtccctgatcgcttcacaggcagtggacctgggacagatttcactctcaccatcagcagtgtgcaggctgaagacctggcagtttattactgcaagcaatcttattatcacatgtatacgttcggatcggggaccaagctggaaataaaacatcatcatca

SEQ ID NO: 9 - DNA, 25 bp, Artificial Sequence
  scFv primer (left)
cagatgcagcagtctggacctgagc

SEQ ID NO: 10 - DNA, 25 bp, Artificial Sequence
  scFv primer (right)
ttatatttccagcttggtccccgat

(19) United States
(12) Patent Application Publication
(10) Pub. No.: US 2009/0288220 A1
(43) Pub. Date: Nov. 19, 2009

(54) Bioreactor using viviparous plant

(76) Inventors: Tai-Hyun Kim, Gyeonggi-do (KR); Yu-Chul Jung, Gyeonggi-do (KR)

Correspondence Address: EDWARDS ANGELL PALMER & DODGE LLP, P.O. BOX 55874, BOSTON, MA, 02205, US

(21) Appl. No.: 11/658,532

(22) PCT Filed: Jan. 20, 2005
(86) PCT No.: PCT/KR05/00177

§ 371 Date: Jul. 6, 2009

(30) Foreign Application Priority Data
Jan. 20, 2004 (KR) 10-2004-0004272

(51) Int. Cl. C12N 15/82; A01H 5/00

(52) U.S. Cl. 800/278; 800/298

(57) ABSTRACT

This invention relates to transgenic plants for producing products of interest such as proteins. Since the transgenic plants according to the invention are cultured in large quantities without culturing tissues and their heredity is preserved through several generations, the invention can yield the products of interest such as proteins in bulk. The invention also provides transgenic plants that are available to the analysis of genomic functions and the production of plants expressing genes by regulating the timing of the expression of the gene of interest by use of proper expression vectors.

### CROSS-REFERENCES TO RELATED APPLICATIONS

This application is a U.S. national phase application, pursuant to 35 U.S.C. §371, of PCT international application Ser. No. PCT/KR2005/000177, filed Jan. 20, 2005, designating the United States and published in English on Aug. 25, 2005 as publication WO 2005/077153 A1, which claims priority to Korean application 10-2004-0004272, filed Jan. 20, 2004. The entire contents of the aforementioned patent applications are incorporated herein by this reference.

### TECHNICAL FIELD

This invention relates to a method for producing target material, for example, protein, antibody and peptide, etc., from transgenic plants. Further, this invention relates to a method for using transgenic plant as bioreactor in order to produce target materials. More specifically, this invention relates to a method for producing interest molecules through successive generations stably and massively, from transgenic viviparous plant which reproduces by vegetative apomixes.

### BACKGROUND

In general, the mass production of biopharmaceuticals has been achieved in microorganisms. For example, a method for producing interest bioactive material, such as, protein, antibody and peptide and etc., from transfected E. coli, Yeast or Fungi was relatively well developed. The microbial system, however, was not suitable to be adopted to produce protein, which would be used as pharmaceuticals, due to the absence of the post-transcriptional process and due to coagulation and the lower solubility of protein in the microbial system. That is, while the 3 dimensional structure of pharmaceutical protein is determined through the post-transcriptional process and thereby the pharmacological activity is determined, the microbial system neither has a modification system nor a system which is different from that of eukaryotes. Thus, the microbial system was not suitable for producing protein having various bioactivities.

Protein expression system employing insect or animal cells was introduced as an alternative system which could provide recombinant mammalian originated proteins having enhanced bioactivities through the post-transcriptional process [see, M A J K, Vine N D. Plant expression systems for the production of vaccines. Curr Top Microbiol Immunol. 236, 275-292 (1999)]. However, the cost of the medium for producing proteins from transgenic insects or animals is very high. Further, there is high risk of animal viral infection. Further, it requires high cost to isolate and purify proteins from the medium. In addition, the mass production of animal cells is not possible with the microbial system, since the cultured animal cells are highly sensitive to the culture conditions.

Since the middle of 1980's, researches using plants have been actively carried out in order to provide an alternative cost effective protein mass production system, and successful results were reported for several plants. Thus, preparing plant-derived products of interest (PPI) from transgenic plants was referred to as “Molecular Farming” or “Biofarming”. The PPI includes pharmaceuticals, such as, protein, antibody, vaccine and other therapeutics; and industrial compounds such as, plastics and oils, etc. The first product from molecular farming was reported in 1989. Molecular farming, which employs a plant as a bioreactor producing interest molecules, such as, protein is considered as an alternative method. The plant system has advantages in time and cost in comparison to the conventional microbial system or animal cell system, since the plant system provides soluble proteins massively with relatively lower cost (for example, about ⅓ of the microbial system and about 1/30 of animal system). Kusnadi, et al [see, Ann R. Kusnadi, Zivko L. Nokolov, John A. Howard (1997) Production of recombinant proteins in transgenic plants: practical considerations. Biotechnol. Bioeng. 56:473-484] reported that the total cost of producing recombinant protein in plant system is just about 1/10 to 1/50 of the cost using E. coli. Thus, protein manufacturing in a plant system has advantages as follows: i) the lower cost of the medium which requires just starches and salts (about 1/10⁴ of the cost of medium for animal system), ii) easy to isolate and purify secreted proteins in the medium, iii) no possibility of animal viral infection. Further, a vector system regulating gene expression using chemical compounds provides a method of controlling the production of a target protein from transgenic plants [see, Hartley et al, 2002, Targeted gene expression in transgenic Xenopus using the binary Gal4-UAS system. Pro. Natl. Acad. Sci. USA 99: 1377-1382].

Until now, about 350 candidate genes have been isolated for study in molecular farming, and several industrial companies are studying various plants for using in molecular farming. Various proteins have been produced from plant such as, tobacco, alfalfa, maze, banana, carrot, potato or tomato. The bioactive molecules obtained from transgenic plants include anticoagulant, thrombin inhibitor, growth hormone, blood substitute, collagen replacement, antimicrobial agent; pharmaceuticals for treating and/or preventing neutropenia; pharmaceuticals for treating and/or preventing anemia; pharmaceuticals for treating and/or preventing hepatitis; pharmaceuticals for treating and/or preventing cystic fibrosis, liver diseases and hemorrhage; pharmaceuticals for treating and/or preventing Gaucher's disease; pharmaceuticals for treating and/or preventing HIV; pharmaceuticals for treating and/or preventing hypertension; and pharmaceuticals for treating and/or preventing organophosphate poisoning, etc.

Even though the plant system has advantages over other systems, the plant system has disadvantages as follows: i) lower growth rate of the bioreactor plant, ii) lower expression rate and productivity of the interest molecule and iii) the requirement of the development of appropriate downstream processes. Therefore, in order for the plant system to be used as an efficient system for manufacturing protein, it requires, i) selection of a plant having rapid growth and showing higher productivity of the interest molecules, ii) development of a potent promoter and a transfection method suitable for the selected plants and iii) development of the technology for optimizing culture conditions and the development of protein purifying method.

Various plant transfection methods have been introduced. The methods are largely divided into two groups: i) transformation of cell or tissue with foreign genes and tissue culturing and ii) in planta transformation which introduces foreign genes providing new genotypes better adapted to biotic and a biotic environmental factor without a tissue culture process.

The transformation of cell or tissue is the most conventional method for transforming plants. This method includes the step of transformation of cell or tissue and the step of culturing the cell or tissue in suitable soils or medium, in order to obtain transgenic plant. This method was well established with tobacco and petunia. The transformation of cell or tissue is carried out by earth microorganism (e.g. Agrobacterium), biolistic gene transfer, PEG-mediated fusion, electroporation or liposome. The co-incubation with agrobacterium, which was used for transforming a dicotyledonous plant, is recently used for transforming a monocotyledon plant. According to this method, a tissue fragment is co-incubated with agrobacterium and then the tissue is differentiated in a re-differentiation medium. Since this method needs the processes of co-incubation, of removal of agrobacterium by the use of antibiotics and of isolation of transformants, the differentiation ability may be damaged to produce no differentiate and the number of transformed plants is significantly reduced through the above-mentioned processes. In order to overcome these problems, a method of plant preculture or using higher pathogenic agrobacterium, which could increase transformation efficiency, was introduced. However, this method did not provide a substantial solution.

Meantime, TMV (Tobacco mosaic virus) or CPMV (cow-pea mosaic virus) can be used as a microorganism instead of an agrobacterium. With regard to biolistic gene transfer, it introduces tungsten or gold molecules coated with DNAs encoding foreign genes using gene guns. It can be used for transforming a dicotyledonous plant, while it is usually used for transforming a monocotyledon plant including graminaceae grasses which cannot be transformed with an agrobacterium. Regarding this method, it is important to establish optimal conditions in consideration of the plant and tissue type; the size and density of the molecule to be bombarded; the amount of DNA and the method of coating; and the velocity and frequency of bombarding. Even if this method can be applied to any type of tissue, it is preferable for this method to use tissues having an active cell dividing activity and an active re-differentiation ability. Various plants, which were successfully transformed by this method using dividing tissue or shoot, were reported. Thus, both agrobacterium co-incubation and molecular bombardment need a regeneration process. Therefore, this method cannot be used for a plant that does not have a well-established re-differentiation process or takes a fairly long time for re-differentiation. On the other hand, the re-differentiated plant often shows somaclonal variation and shows the problem of genetic stability. Therefore, it should be investigated thoroughly whether or not the undesired genetic mutation resulted from the tissue culture process. If the mutation is induced during the tissue culture process, the mutation inducing step should be clearly detected and suitable ways for minimizing or inhibiting the mutation should be made. If the mutation is a result of intact mutation, an appropriate method for selectively prohibiting re-differentiation of the mutant cells should be introduced. Thus, the need for minimizing cell mutation requires an alternative plant transformation method which could remove or minimize the step of tissue incubation by introducing foreign genes into the tissue fragment without in vitro incubation.

In-planta transformation was introduced as a method for obtaining transformed plants without tissue culture and regeneration processes. According to this method, transformed seeds or adventitious roots are obtained from differentiating stem from transformed cells after the cells are transformed on the growing point or meristem. As a method for transforming meristem such as, vacuum infiltration method, floral meristem dipping method and agrobacteria spraying were developed for this method. This method was well established in Arabidopsis. In this method, agrobacterium is introduced to meristem in pollen of a plant followed by identifying transformants by culturing the seeds obtained from the plant. If the T-DNA of agrobacterium is introduced into the chromosome of a reproductive cell, then the transformants can be identified in the next generation. As an alternative method by not using agrobacterium, a method applying foreign DNAs on the style of a pollinated flower was developed with a rice plant and tobacco in 1992 [see, Langridge, P. et al. (1992) Transformation of cereals via Agrobacterium and the pollen pathway: a critical assessment, Plant J. 2:631-638]. In this method, transformed seeds are obtained by introducing DNAs directly to the stigma of a pistil after cutting the stigma, wherein the stigma has a pollen tube pathway.

Thus, the conventional plant transformation methods can be applied only to a limited number of plants; which have problems of inconvenient processes of transformation and tissue culturing; and which have problems of somatic cell mutation during re-differentiation and regeneration processes; and which have a problem of a reduced rate of occurrence of transformed plants in the next generation. Thus, the conventional methods do not provide effective bioreactors in order to produce protein massively. Therefore, the need of finding a new plant, which can be used in transformation, and the need of developing an efficient plant transformation system still continue.

### DESCRIPTION OF THE INVENTION

This invention relates to a transformed plant for producing interest molecules such as protein. In this invention, the transformed plants are cultured massively without a tissue culture process, and the genetic stabilities of the transformed plants surprisingly continued through to several following generations. Therefore, it is possible to produce interest molecules, such as proteins, massively with the present invention. Further, this invention can be used as an important tool for the analysis of gene function and for obtaining transformed plant expressing foreign genes by regulating the expression using suitable expression vector.

Thus, the object of this invention is to provide a method for transforming an asexually reproducing plant using genetic material. Specifically, this invention provides a in vivo transformation method by using a viviparous plant which produces vegetative apomixes.

Further, the object of this invention is to provide a transformed viviparous plant, which is used as a bioreactor for producing interest molecules such as protein.

Further, the object of this invention is to provide a method for producing interest molecules from the transformed viviparous plant reproducing by vegetative apomixes.

In order to achieve the objects, we, the inventors selected a perennial viviparous plant having a large biomass. The perennial viviparous plant reproducing asexually is characterized by propagating through a completely differentiated progeny plant, plantlets, bulbils or gemmae. We, inventors, confirm transformed progenies after introducing DNAs encoding foreign genes expressing interest molecules.

In an embodiment, Kalanchoe or Bryophyllum belonging to the Crassulaceae family were used as a viviparous plant. Firstly, leaves in full growth, which do not have plantlets, were selected and gathered with their petioles. Then, the gathered leaves were scratched for 5 times to 10 times with tungsten pin (diameter of 0.2 mm) at the serrated edges of the leaves where the plantlet would be generated. After 3 to 5 minutes from the scratching, 1 or 2 drops of agrobacterium suspension were applied to the scratched area, and then the leaves were incubated at 25° C. under 1,500 lux of light for 5 to 10 days. Then, asexually reproduced leaflets, which were developed on the serrated edges of the treated leaves, were collected in order to find out the transformants. Thus, in situ introduction of a foreign gene into the site where the leaflets would develop resulted in transformed generation. It shows that transformed plants can be obtained without further tissue culture, regeneration and re-differentiation in the present invention.

In another embodiment, plantlets isolated from the parental plant were transformed in situ. The naturally developed off-springs (plantlets) (10˜15 mm in length) resulted from asexual reproduction were isolated from the field-cultured plants. The isolated plantlets were moved into a well-closed container and were cultured at 25° C. for 20˜30 hrs in a dark room while providing enough water to maintain the stomatal spore openings, and then the cultured off-springs were submerged in agrobacterium suspension in a glass beaker. Next, 150˜250 μl/L of Silwet L-77 (catalog# vis-01) (registered trademark) was added to the suspension, followed by applying 400 mmHg of pressure for about 30 minutes in order to maintain a vacuum. After 30 minutes from the beginning of applying pressure, the pressure was rapidly removed. Subsequently, the plantlets were transferred to 3 MM paper, and were cultured at 25° C. for 20˜30 hrs. The obtained normal off-springs were used in the next experiments.

In another embodiment, it was confirmed that the off-springs (plantlets) developed from the transformed parental plant have the same genotypes as the transformed parental plant.

An introduction of desired genes was investigated with a GFP fluorescence assay and a PCR method (genomic PCR and RT-PCR). The expression of fluorescence of introduced GFP was detected with a human eye after irradiating UV light (380 nm) using a UV lamp in a dark room. Each of the plantlets confirmed as expressing GFP was transplanted in their to respective pots, which was numbered individually, and the plantlets were cultured to develop next generations. According to the method mentioned above, T₁ (the second generation) and T₂ (the third generation) generations were cultured and confirmed. The expression of fluorescence of introduced GFP was detected with a human eye after irradiating UV light (380 nm) using UV lamp, in a dark room like the above. Each of the plantlets confirmed as expressing GFP was transplanted into their respective pots, which was numbered individually, and the plantlets were cultured to develop into the following generations, T₁ (the second generation) and T₂ (the third generation) following the method mentioned above. Further, the introduction of the interest genes was detected using a con-focal microscope under the irradiation of UV light (460 nm). Further, the introduction of a gene was confirmed by the carrying out of PCR and RT-PCR.

In another embodiment, a plant was transformed using a GUS gene and the protein expression was detected by dying a GUS protein with X-Glu in four successive generations.

In another embodiment, the expression of scFv antibody was assayed using genomic PCR, RT-PCR and western blot, and the activities thereof were detected in comparison to those obtained from E. coli.

### BRIEF DESCRIPTIONS OF DRAWINGS

FIG. 1 shows the cleaved construct of pCAMBIA1303 vector.

FIG. 2a shows a picture of the first generation of transgenic plants (T₀) under confocal microscope.

FIG. 2b shows the second generation of transgenic plant (T₁) under confocal microscope.

FIG. 2c shows the third generation of transgenic plant (T₂) under confocal microscope.

FIG. 3a represents the picture of electrophoresis for genome PCR results using GUS primers.

FIG. 3b represents the picture of electrophoresis for genome PCR results using mGFP5 primers.

FIGS. 4a, 4b and 4c represent the pictures of electrophoresis for genome RT-PCR results using a GUS primer.

FIG. 5 shows the construct of a vector for a GUS transformation.

FIGS. 6a and 6b represent the results of X-Glu dyeing of a GUS protein expressed by transformation.

FIG. 7 shows the construct of a vector for scFv transformation.

FIG. 8a represents the result of a transformation of scFv antibody.

FIG. 8b represents the activity of scFv antibody

This invention will be described in more detail by the examples given below. However, it is intended that the examples are considered exemplary only and the scope of the invention is not limited thereto.

### EXAMPLE

### Example 1

### Plants Used in this Invention

Among the plants reproduced by vegetative apomixes, K. pinnata, K. daigremontianum and K. tubiflora, which belong to Kalanchoe or Bryphyllum genus, were selected for this experiment. K. pinnata, K. daigremontianum and K. tubiflora were from Madagascar in North Africa. They were cultured for not more than 3 months to have a length of about 20 cms measured from the earth in a culture room maintaining constant room temperature and constant humidity, before they were used in this experiment.

### Example 2

### Plant Transformation by Vacuum Infiltration

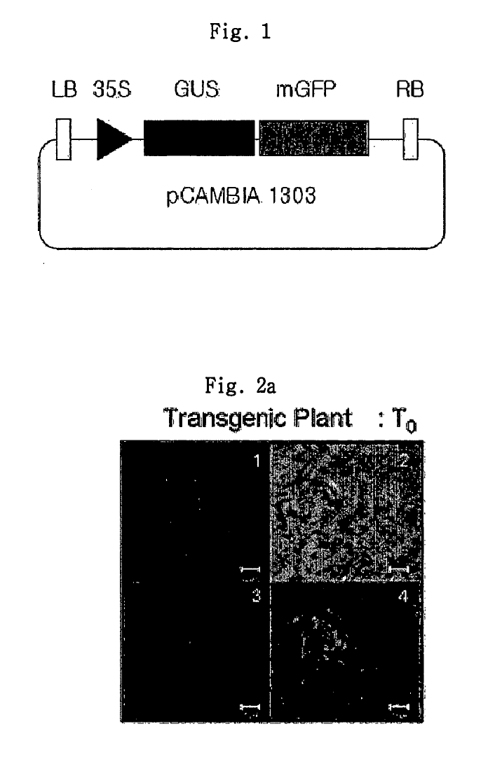

Plantlets being about 10 cms in length were removed from the edges of the plants of example 1. pCAMBIA1303 vector (Center for Application of Molecular Biology to International Agriculture was employed to introduce foreign DNAs) (FIG. 1) (SEQ. ID. NO.: 1). The pCAMBIA1303 vector included a hygromycin resistant gene and a Kanamycin resistant gene as resistant genes, and included GUSA:GFP as selection markers. The pCAMBIA1303 vector was suitable to detect whether or not the interest gene was introduced, since it had two (2) reporter genes and it had broad antibiotic applications. Agrobacterium (LBA4404) having a pCAMBIA1303 vector was mixed cultured in YEP medium (500 ml) for two (2) days at 27° C. Then, the cultured Agrobacterium was transferred to a tube for centrifugation. Agrobacterium was removed from the medium by the carrying out of a centrifugation for 15 minutes with 2,500 rpm. The removed agrobacterium was moved to MS medium (200 ml) comprising 0.5 g/l of MES. 200 μl/L of Silwet (catalog# vis-01) (registered trademark) was added to the obtained suspension. Then, plantlets having roots, which were formed when the plantlets were developed in the parental leaf, were submerged into the suspension followed by applying 400 mmHg of pressure. After 30 minutes from the beginning of applying pressure, the pressure was rapidly removed. Subsequently, the plantlets were transferred to 3 MM paper in a petridish and were incubated at about 25° C. for 1 day under dark conditions. After 1 day of incubation, newly developed leaves having normal shapes were transferred to a pot.

### Example 3

### Plant Transformation by Pin Prickle Method

Agrobacterium culture medium made in example 2 was used in this experiment. Stress was applied to the edges of the fully-grown leaves of plants of example 1, using tungsten pin. After applying the culture medium to the edges, the leaves were incubated at 25° C. in light a culture device until new plantlets were developed. After about 1 week, new plantlets developing roots were transplanted to a pot.

### Example 4

### Detection of Transformation

i) GFP Detection

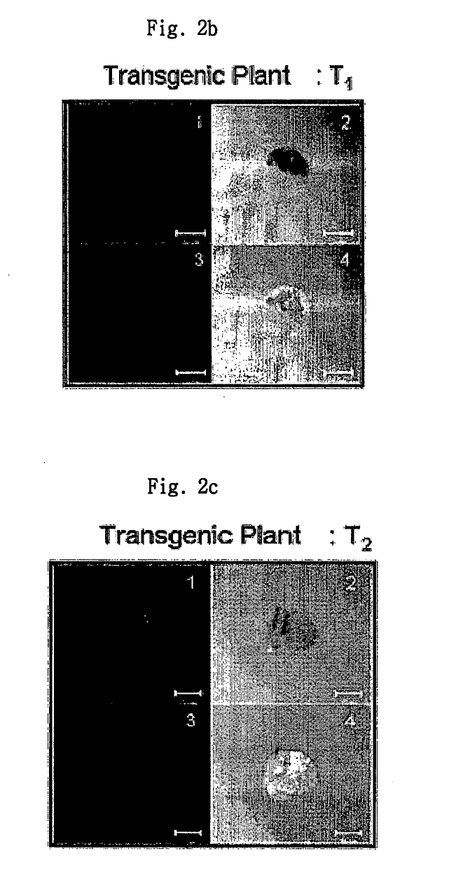

The expression of fluorescence protein of introduced GFP was detected with a human eye after irradiating UV light (380 nm) using a UV lamp in a dark room. Each of the plantlets confirmed as expressing GFP was transplanted into respective pots, which were individually numbered, and the plantlets were cultured to develop the following generations. According to the method mentioned above, T₁ (the second generation) and T₂ (the third generation) generations were cultured and confirmed. The expression of fluorescence of introduced GFP was detected with eye after irradiating UV light (380 nm) using a UV lamp in a dark room as mentioned above. Each plantlet expressing GFP was transplanted to respective pots, which were numbered individually, and the plantlets were cultured to develop to next generation, T₁ (the second generation) and T₂ (the third generation) following the method of the above mentioned. Further, the introduction of the interest genes was detected using confocal microscope under conditions of UV light (460 nm) irradiation. FIGS. 2a, 2b and 2c show the detection results with K. pinnata, wherein section 1 means UV light, section 2 means background, section 3 means normal visible light, and section 4 means mixed light, respectively.

ii) Carrying Out PCR (Genomic PCR)

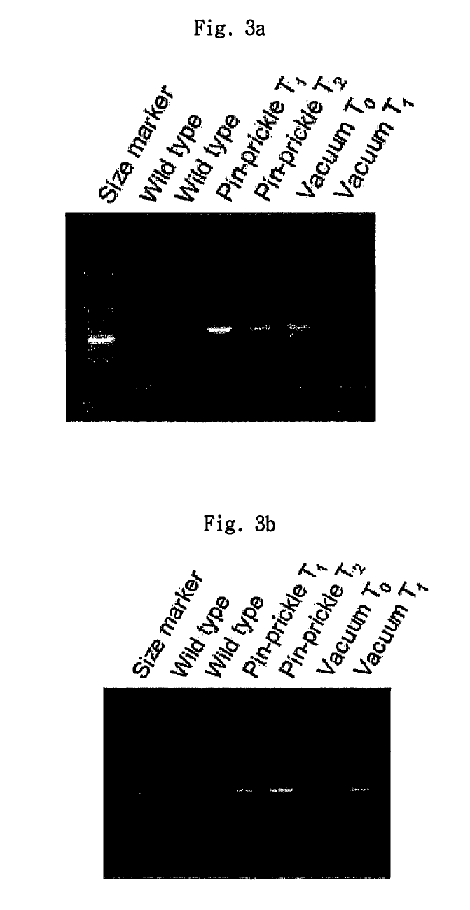

The introduction of the interest genes was detected with genomic PCR and RT-PCR. First, genomic DNAs were extracted using lysis buffer solution. The extracted genes were treated with BamHI and HindIII, and were reacted at 37° C. for 45 minutes in a constant temperature water bath followed by a successive reaction at 37° C. for 3 hours in a constant temperature water bath. PCR was carried out using 3 μl˜5 μl of the digested genomic DNAs and GUS primer [left: ctgatagcgcgtgacaaaaa (SEQ. ID. NO.: 2) and right: ggcacagcacatcaaagaga (SEQ. ID. NO.: 3)] and GFP primer [left: tcaaggaggacggaaacatc (SEQ. ID. NO.: 4) and right: aaagggcagattgtgtggac (SEQ. ID. NO.: 5)] with adding 5 μl of distilled water and 10 μl of PCR-premix. PCR was carried out under the following conditions: i) 10 minutes at 95° C., ii) 30 seconds at 94° C., iii) 30 seconds at 56° C., iv) 30 seconds at 72° C. followed by carrying out 30 cycles of ii) to iv) processes and 10 minutes at 72° C. FIG. 3a shows the PCR result for K. pinnata, using GUS primer, and FIG. 3b shows the PCR results for K. pinnata, using mGFP5 primers.

iii) Carrying Out RT Reverse Transcription)-PCR

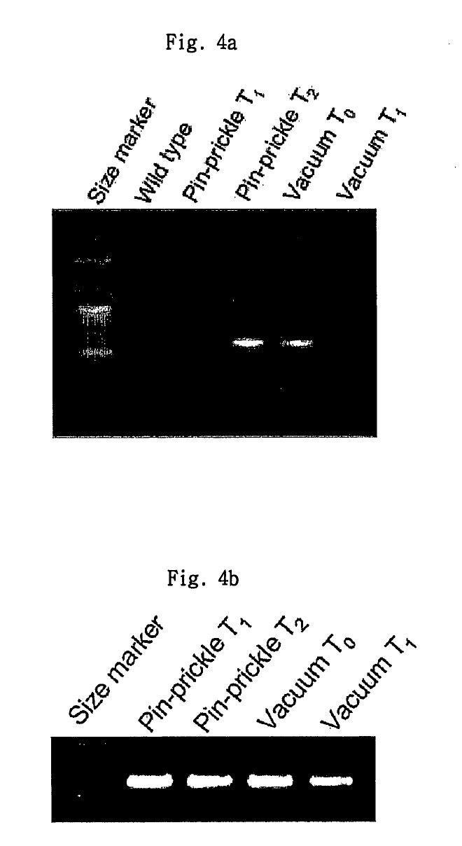

The total RNA of a plant was extracted according to a conventional hot-extraction method [see, T. C. Verwoerd, B. M. Dekker, and A. Hoekema (1989) A small-scale procedure for the rapid isolation of plant RNAs. Nucl. Acids. Res 17: 2362]. Target tissue was rapidly freezed with liquid nitrogen and was grounded in a pastle, and 2 ml of the grounds was moved to E-tube. Subsequently, 500 μl of extraction buffer [penol: 0.1 M LiCl, 100 mM of Tris-HCl, pH=8.0, 10 mM of EDTA, 1% SDS (1:1)], which was heated at about 80° C., was added to the tube and the mixture was agitated. The mixture was agitated again after adding 250 μl of chloroform-isoamylalchol (24:1). After centrifugation at 12,000 rpm for 5 minutes, the supernant was moved to a tube. Then, the same amount of 4 M LiCl was added to the tube. After reaction for 14 hours at room temperature, centrifugation was carried out at 12,000 rpm for 10 minutes, and the precipitates were collected while removing the supernant. The obtained precipitates were solved in distilled water treated with 150 μl of diethyl pyrocabonate (DEPC) and then a 0.1 volume of 3 M sodium acetate and a second time of the total volume of 100% ethanol were additionally added to the mixture followed by reaction at −4° C. freezer for 3 hours. Subsequently, precipitates were obtained after centrifugation at 15,000 rpm for 30 minutes, and then the obtained precipitates were dissolved in 50 μl of DEPC treated distilled water and the solution was stored at −70° C. in a freezer. The concentration of the purified total RNA was detected using a spectrum analyzer. 5 μg of the total RNA was diluted using DEPC treated distilled water to have a total volume of 10.5 μl in a 0.5 ml E-tube. Then, 3.0 μl of 10 pM oligo-dT was added and the mixture was heated to 70° C. for 10 minutes using PCR thermocycler (PTC-0200, MJ Research). After cooling the mixture at 4° C., 6.0 μl of 2.5 mm dNTPs and 5.0 μl of 5× reaction buffer solution was added. Subsequently, the mixture was put into reaction at 37° C. for 10 minutes, and then was cooled to 4° C. Then, 0.5 μl of 200 U/μl reverse transcriptase was added and a reaction was carried at 37° C. for 1 hour. After synthesizing cDNA following the reaction at 70° C. for 10 minutes, the cDNAs were stored at 4° C. 3.0 μl of the synthesized cDNAs, respective 1.0 μl of 5′ part and 3′part of a 10 pM gene specific primer, 2.5 μl of 2.5 mM dNTPs, 10 μl of sterilized distilled water, 2.0 μl of 10× reaction buffer solution and 0.5 μl of tag synthetase were added and PCR was carried out using (PTC-0200, MJ Research). GUS primer [left: ctgatagcgcgtgacaaaaa (SEQ. ID. NO.: 2) and right: ggcacagcacatcaaagaga (SEQ. ID. NO.: 3)] and GFP primer [left: tcaaggaggacggaaacatc (SEQ. ID. NO.: 4) and right: aaagggcagattgtgtggac (SEQ. ID. NO.: 5)] were used in PCR. PCR was carried out under the following reaction conditions: i) 10 minutes at 95° C., ii) 30 seconds at 94° C., iii) 30 seconds at 56° C., iv) 30 seconds at 72° C. followed by carrying out repeated 30 cycles of ii) to iv) processes and 10 minutes at 72° C. FIGS. 4a to 4c show the PCR result for K. pinnata, using a GUS primer, and FIG. 3b shows the PCR results for K. daigremontianum and K. tubiflora and K. pinnata, using a GUS primer. The detection results using GFP and GUS genes represent that the plants in this invention stably expressed the foreign genes in their next generations and the following generations (T₁ and T₂). The tables 1 and 2 show transformation rates using a vacuum insertion method and pinprickle method, respectively.

Table 1: Transformation Rates Using a Vacuum Insertion

TABLE 1
Transformation rates using a vacuum insertion

| | K. pinnata | K. daigremontianum | K. tubiflora |
|---|---|---|---|
| P | 150 | 150 | 150 |
| T₀*/P | 109.95/150 | 109.20/150 | 93.48/150 |
| (Efficiency %) | (73.30%) | (72.80%) | (62.32%) |
| T₁*/T₀* | 145.25/150 | 145.53/150 | 144.66/150 |
| (Efficiency %) | (96.83%) | (97.02%) | (96.44%) |

P: The number of plantlets used in the transformation
T₀*: The number of first transformed generations
T₁*: The number of second transformed generations

TABLE 2
Transformation rates using a pinprickle method

| | K. pinnata | K. daigremontianum | K. tubiflora |
|---|---|---|---|
| P | 150 | 150 | 150 |
| T₀*/P | 126.24/150 | 121.11/150 | 116.73/150 |
| (Efficiency %) | (84.16%) | (80.74%) | (77.82%) |
| T₁*/T₀* | 148.35/150 | 146.43/150 | 145.52/150 |
| (Efficiency %) | (98.90%) | (97.62%) | (97.01%) |

P: The number of plantlets used in the transformation
T₀*: The number of first transformed generations
T₁*: The number of second transformed generations

### Example 5

### Expression of GUS Protein

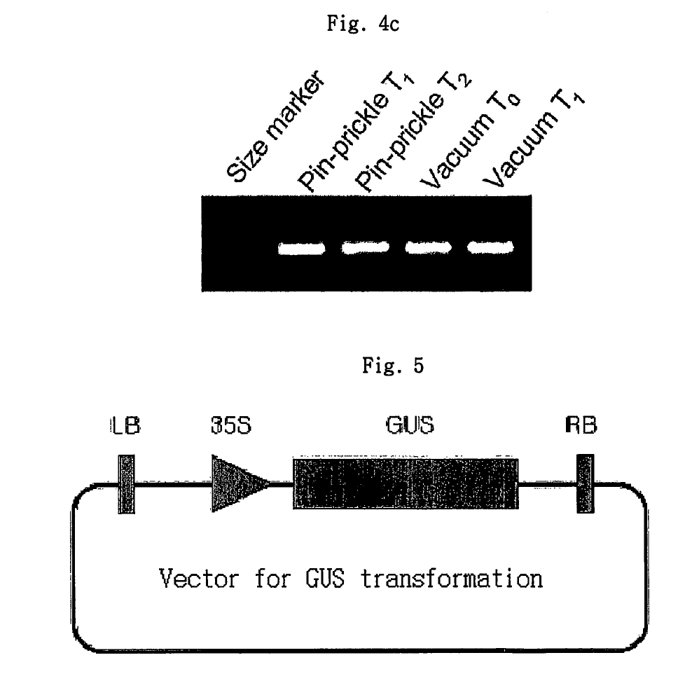

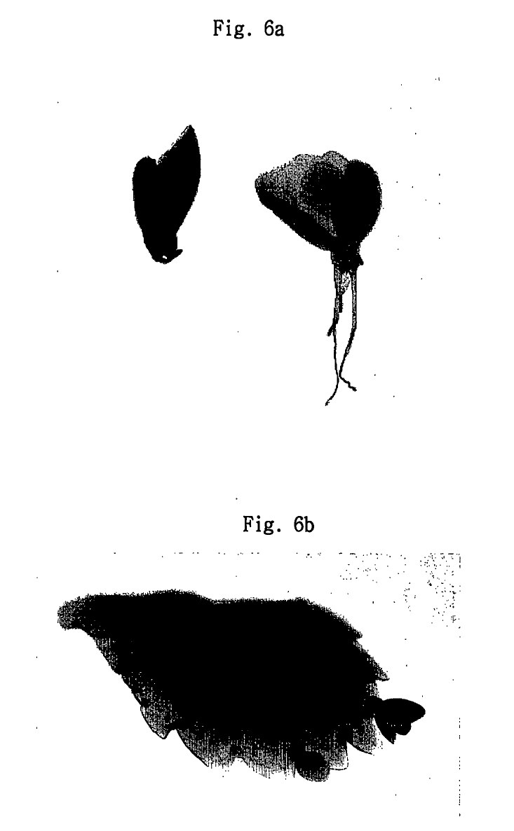

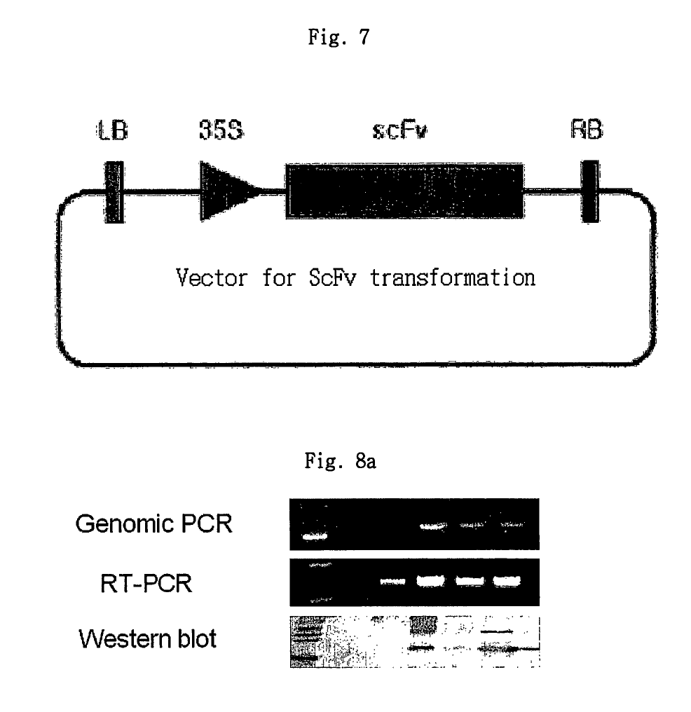

Except introducing GUS gene (SEQ. ID. NO.: 6) into the vector in example 2 (see FIG. 5), a plant was transformed in the same way in examples 1, 2 and 3 and the protein expression was detected by dying a GUS protein with X-Glu. The tissue of K. pinnata was put into a priory cooled 90% acetone and was stored on ice for 20 minutes, and then the acetone on the surface of the tissue was removed with a paper towel. Then, the plant was moved to a X-Glu dyeing solution comprising 0.1% Triton, 50 mM NaPO₄, 2 mM ferricyanide, 2 mM ferrocyanide and 10 mM EDTA. The tissue was soaked with the dyeing solution for 30 minutes under a vacuum condition by a vacuum pump. Next, the tissue in the dyeing solution was in reaction at 37° C. in an incubator for 8 hours. After dyeing, the plant was treated with 70% alcohol in order for the control tissue to be bleached to have a white color. Referring to FIG. 6a, the left of the picture represents a blue colored plant (third generations of transgenic plant) which expressed GUS, and the right represents untransgenic plant. Further, in order to detect a transition of a GUS protein expression from a parental plant to a progeny, the whole parental plant having progenies was dyed. In FIG. 6b, the plantlets were developed from the edge of the parental plant's serrated leaf and the protein was expressed in the plantlet at the same time. Such protein expression was detected till the fourth generation.

### Example 6

### Expression of scFv Antibody and its Activity

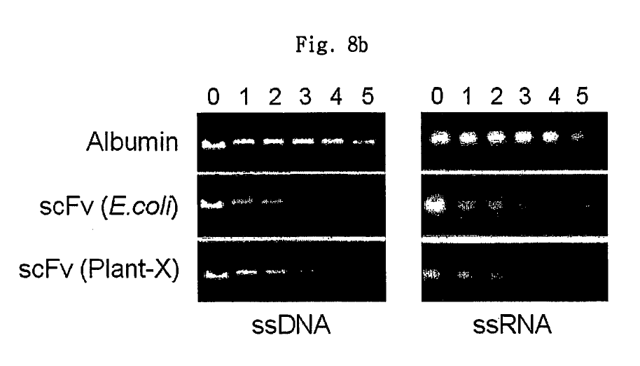

Except introducing scFv genes (SEQ. ID. NO.: 7 and SEQ. ID. NO.: 8) into the vector in example 2 (see FIG. 5), a plant was transformed in the same way in examples 1, 2 and 3 and an antibody expression was detected and a protein expression and its activity were detected. K. pinnata of the example 1 was transformed, and each sample of the developed generations was collected. Then, genomic DNA and RNA were extracted from each sample and the introduction of the genes was determined. In order to carry out a genomic PCR, the genomic DNAs were extracted with genomic PCR lysis buffer. The extracted genomic DNA was treated with BamHI and HindIII. A reaction of the treated DNAs was carried out at 37° C. for 45 minutes in a constant temperature water bath followed by an additional reaction at 37° C. for 3 hours in a constant temperature incubator. After adding 3 μl˜5 μl of cleaved genomic DNAs and scFv primer [left: 5′cagatgcagcagtctggacctgagc3′(SEQ. ID. NO.: 9)] and [right: 5′ttatatttccagcttggtccccgat3′(SEQ. ID. NO.: 10)], PCR was carried with 5 μl of distilled water and 10 μl of PCR-premix. PCR was carried out under the following reaction conditions: i) 10 minutes at 95° C., ii) 30 seconds at 94° C., iii) 30 seconds at 56° C., iv) 30 seconds at 72° C. followed by a carrying out of repeated 30 cycles of ii) to iv) processes and 10 minutes at 72° C. The total RNAs were extracted in order to carry out RT (Reverse transcription)-PCR according to the conventional hot-extraction method (see, T. C. Verwoerd, B. M. Dekker, and A. Hoekema (1989) A small-scale procedure for the rapid isolation of plant RNAs. Nucl. Acids. Res 17: 2362). A target tissue was rapidly freezed with liquid nitrogen and was ground in a pastle, and 2 ml of grounds was moved to E-tube. Subsequently, 500 μl of an extraction buffer [penol: 0.1 M LiCl, 100 mM of Tris-HCl, pH=8.0, 10 mM of EDTA, 1% SDS (1:1)], which was prior heated about 80° C., was added to the tube and the mixture was agitated. The mixture was agitated further after adding 250 μl of chloroform-isoamylalchol (24:1). After centrifugation at 12,000 rpm for 5 minutes, the supernant was moved to a tube. Then, the same amount of 4 M LiCl was added to the tube. After a reaction for 14 hours at room temperature, centrifugation was carried out at 12,000 rpm for 10 minutes, and the precipitates were collected while removing the supernatant. The obtained precipitates were solved in distilled water treated with 150 μl of diethyl pyrocabonate (DEPC) and then 0.1 volume of 3 M sodium acetate and a second time of the total volume of 100% ethanol was additionally added followed by reaction at −4° C. freezer for 3 hours. Subsequently, the precipitates were obtained after centrifugation at 15,000 rpm for 30 minutes, and then the obtained precipitates were solved in 50 μl of DEPC treated in distilled water tot −70° C. in a freezer. The concentration of purified total RNA was detected with a spectrum analyzer. 5 μg of the total RNA was diluted using a DEPC treated distilled water to have total volume of 10.5 μl in a 0.5 ml E-tube. Then, 3.0 μl of 10 pM oligo-dT was added and the mixture was heated to 70° C. for 10 minutes using PCR thermocycler (PTC-0200, MJ Research). After cooling the mixture at 4° C., 6.0 μl of 2.5 mm dNTPs and 5.0 μl of 5× reaction a buffer solution was added. Subsequently, the mixture was put into reaction at 37° C. for 10 minutes, and then was cooled to 4° C. Then, 0.5 μl of 200 U/μl reverse transcriptase was added and reaction was carried at 37° C. for 1 hour. After synthesizing cDNA from the reaction at 70° C. for 10 minutes, the cDNAs were stored at 4° C. 3.0 μl of the synthesized cDNAs, respective 1.0 μl of 5′ part and 3′part of 10 pM gene specific primer, 2.5 μl of 2.5 mM dNTPs, 10 μl of sterilized distilled water, 2.0 μl of 10× reaction buffer solution and 0.5 μl of tag synthetase were added and PCR was carried out using (PTC-0200, MJ Research). The scFv primers of [left: 5′ cagatgcagcagtctggacctgagc 3′ (SEQ. ID. NO.: 9)] and [right: 5′ ttatatttccagcttggtccccgat 3′ (SEQ. ID. NO.: 10)] were used in PCR. The PCR was carried out under the following reaction conditions: i) 10 minutes at 95° C., ii) 30 seconds at 94° C., iii) 30 seconds at 56° C., iv) 30 seconds at 72° C. followed by carrying out repeated 30 cycles of ii) to iv) processes and 10 minutes at 72° C. Next, in order to carry out westernblotting, the total proteins of the plant were isolated with heat protein isolation method. The isolated proteins were subject to SDS-PAGE electrophoresis, and the electrophoresis gel was transited to nylon membrane in a transfer buffer solution (25 mM Tris-Cl, pH 8.3, 1.4% glycin, 20% methanol). The membrane was submerged into TBST buffer solution comprising 1% bovine serum albumin (10 mM Tris-Cl, pH 8.0, 150 mM NaCl, 0.05% Tween 20®) and the solution was shaken for 1 hour at room temperature. Next, the membrane was rinsed three times (each time for 10 minutes) with a clean TBST solution. The membrane was fully submerged by adding scFv antibody dilute. Then, after a reaction at 4° C. for 1 hour, the membrane was additionally rinsed three times (each time for 10 minutes) with clean TBST solution. Next, the membrane was reacted with goat anti-rabbit IgG-alkaline phosphatase which was diluted with a TBST solution. After a reaction for 30 minutes, the membrane was rinsed three times (each time for 10 minutes) with TBST solution. The membrane was submerged into an alkaline phosphatase substrate solution and was mildly shaken to develop desired protein band. In FIG. 8a, lane 1 represents a size marker, lane 2 represents a negative control, lane 3 means a plant of 0 generation (parental plant), lane 4 means a plant of first generation, lane 5 means a plant of second generation and lane 6 means a positive control, respectively. It was confirmed that genes were stably expressed in all detected generations.

Then, the activity of the expressed antibody of scFv was detected. An scFv was isolated using IgG-sepharose affinity chromatography according to its affinity to ssDNA. The isolated scFv was located at about 32 kDa portion in 10% acrylamide gel. The ssRNA was prepared by the sub-cloning of TMV coat protein gene into LITMUS vector (New England Biolabs). The LITMUS vector having TMV coat protein gene was isolated in linear form by treating the vector with Stu I. An ssRNA was treated with 20 μl of reaction mixture comprising 5 μl of LITMUS vector, 5 μl of 10× buffer solution, 2 μl of 100 mM DTT, 4 μl or 2.5 mM rNTP and 1 U T7 RNA polymerase in a tube. After incubating the mixture at 37° C. for 3 hours, 1 U DNase was added thereto. Subsequently, the ssRNA was incubated at 37° C. for 20 minutes, and then the results of transcription were analyzed in 1% agarose gel. The DNase and RNase analysis reaction was carried out. Both of DNA (0.25 μg) and RNA (0.25 μg) were added to a buffer solution (pH 8.0) comprising 20 mM Tris-HCl, 50 mM NaCl and 5 mM MgCl₂. The activities were analyzed by the use of agarose gel electrophoresis at every 0, 1, 2, 3, 4 and 5 hours, after the reaction between the solution and the scFv which was expressed from E. coli. Likewise, the activities were analyzed by the use of agarose gel electrophoresis at every 0, 1, 2, 3, 4 and 5 hours, after the reaction between the solution and the scFv which was expressed from Kalanchoe. In comparison to the negative controls using albumin treated specimen, the scFv prepared according to this invention showed ssDNA and ssRNA lysis activity like the scFv obtained from E. coli (FIG. 8b).

### INDUSTRIAL APPLICATION

Thus, in accordance with this invention characterized by transforming asexually reproducing plants, it is possible to introduce genes expressing interest proteins into a plant with less gene mutation rate. Therefore, it is possible to produce interest molecules, such as protein, massively and cost effectively in comparison to the conventional methods such as using a microbial system or an animal cell system.

1. A method for preparing a transgenic viviparous plant comprising: i) culturing a viviparous plant reproducing by vegetative apomixes; ii) introducing a DNA or RNA encoding a target molecule into the portion where a plantlet would develop; iii) obtaining a plantlet by culturing the viviparous plant of ii); and iv) incubating the plantlet.

2. A method for preparing a transgenic viviparous plant comprising: i) culturing a viviparous plant reproducing by vegetative apomixes; ii) isolating a developing plantlet from the plant; iii) introducing a DNA or RNA encoding a target molecule into the plantlet; and iv) culturing the plantlet of iii).

3. The method of claim 1, wherein the viviparous plant is Kalanchoe or Bryophyllum genus.

4. A method of preparing a target molecule, comprising: i) culturing a viviparous plant reproducing by vegetative apomixes; ii) introducing a DNA or RNA encoding a target molecule into the portion where plantlet would develop; iii) obtaining a plantlet by culturing the viviparous plant of ii); iv) culturing the plantlet; and v) isolating and purifying the target molecule from the transformed viviparous plant.

5. A method for preparing a target molecule, comprising: i) culturing a viviparous plant reproducing by vegetative apomixes; ii) isolating a developing plantlet from the plant; iii) introducing a DNA or RNA encoding a target molecule into the plantlet; iv) obtaining a transgenic plant by culturing the plantlet of iii); and v) isolating and purifying the target molecule from the transgenic viviparous plant.

6. A target molecule prepared in accordance with the method of claim 4.

7. The method of claim 4, wherein the viviparous plant is Kalanchoe or Bryophyllum genus.

8. A transgenic viviparous plant prepared by the method of claim 1.

9. The transgenic viviparous plant of claim 8, wherein the viviparous plant is Kalanchoe or Bryophyllum genus.

10. The method of claim 2, wherein the viviparous plant is Kalanchoe or Bryophyllum genus.

11. A target molecule prepared in accordance with the method of claim 5.

12. The method of claim 5, wherein the viviparous plant is Kalanchoe or Bryophyllum genus.

13. A transgenic viviparous plant prepared by the method of claim 2.
